(19) United States
(12) Patent Application Publication
(10) Pub. No.: US 2017/0266130 A1
(43) Pub. Date: Sep. 21, 2017

(54) COMPOSITIONS, METHODS FOR MAKING THE COMPOSITIONS, AND METHODS FOR TREATING JOINT DISEASE

(71) Applicant: Script Essentials, LLC, Broomfield, CO (US)

(72) Inventor: Suzy Cohen, Broomfield, CO (US)

(73) Assignee: Script Essentials, LLC, Broomfield, CO (US)

(21) Appl. No.: 15/391,462

(22) Filed: Dec. 27, 2016

Related U.S. Application Data
(60) Provisional application No. 62/270,129, filed on Dec. 21, 2015.

(51) Int. Cl. A61K 31/10; A61K 9/48; A61K 9/20; A23L 33/10; A61K 38/39; A61K 36/324; A61K 31/728; A61K 36/906; A61K 9/00; A61K 33/04

(52) U.S. Cl. CPC A61K 31/10; A61K 9/0053; A61K 9/48; A61K 9/20; A61K 33/04; A61K 38/39; A61K 36/324; A61K 31/728; A61K 36/906; A23L 33/10; A23V 2002/00

(57) ABSTRACT

The present disclosure relates to a compound, method of making and method of using such compound preferably in the form of a dietary supplement. The composition, when administered, is capable of treating arthritis and various joint-related pain syndromes. The unique combination of the composition is preferably administered orally. A preferred composition is comprised of at least 10 to 200 mcg of selenium, from about 200 to 1200 mg of MSM, from about 100 to 900 mg of boswellia, from about 1 to 5 mg of type II collagen, from about 25 to 200 mg of hyaluronic acid, and from about 50 to 500 mg of ginger.

### CROSS REFERENCE TO RELATED APPLICATIONS

This application claims the benefit of U.S. Provisional Patent Application No. 62/270,129, filed Dec. 21, 2015, the entire disclosure of which is incorporated herein by reference.

### FIELD OF THE INVENTION

The present invention is directed to compounds, particularly dietary supplements, and methods for formulating and administering the same.

### BACKGROUND OF THE INVENTION

There are at least 360 joints in the human body, which are repeatedly used during the course of a typical day. Inflammation in these joints can and often does occur. “Arthritis” often results from such inflammation, which may result in pain and reduced range of motion.

Arthritis is the number one cause of disability in the United States. More than 50 million adults have been diagnosed with some form of arthritis, which amounts to one in five people over the age of 18. Although proper diet and performing regular exercise can alleviate problems such as arthritis, simply maintaining a healthy lifestyle doesn't eliminate inflammation of joints or the problems associated therewith.

Over time, and particularly as the human body ages, joints in a person's body may decline and lose optimal function. This “wear and tear” on human joints often leads to a condition called “osteoarthritis.” Osteoarthritis is the most frequent cause of joint pain experienced by the over 50 million adults diagnosed with arthritis. Osteoarthritis starts with a breakdown of the cartilage matrix, later progressing to erosion of the cartilage in the joint and eventually release of collagen fragments. During a chronic inflammatory response, the body releases cytokines in response to this breakdown of the cartilage matrix or erosion of the cartilage in the joint. When the cytokine response is chronic and excessive, these well-intended compounds can cause further pain and inflammation (because they are no longer in normal levels, rather they are in excessive amounts).

Tendons are a type of connective tissue that assist in joint movement and maintain joint flexibility. Ligaments are tissues that attach bone to bone. Elastin, a type of connective tissue, makes up the majority of most ligaments. When experiencing arthritis and other degenerative joint diseases, it is common for a person's collagen and elastin to become injured by inflammation, which further contributes to joint immobility. Common joint-related pain often leads to swelling, pain, stiffness and decreased range of motion. Symptoms can be intermittent, and pain often ranges from mild, moderate or severe. As joints, cartilage and connective tissue begin to deteriorate, chronic pain sets in, often making it difficult for the person to perform daily activities. The changes in a person's joints may be visible, for example, swollen knobby finger joints. Most often, joint damage and inflammation shows up only on imaging scans, such as x-rays.

Some forms of arthritis are hereditary. Other types of arthritis are infectious or driven by food allergies or dietary consumption. The pain and inflammation may spread beyond the joint pocket and affect the whole body, such as the heart, eyes, lungs, kidneys and even skin. These types of infectious forms of arthritis are sometimes driven by the immune system, termed auto-immune arthritis, or rheumatoid arthritis, and are also common in adults.

There presently exist many unresolved problems relating to arthritis, and joint-related disorders in particular. By way of example, insufficient amounts of collagen can contribute to joint stiffness and pain. Collagen is the major component of connective tissue in the human body, and is found in skin, muscle and tendons.

Despite current treatment interventions, people with arthritis or joint-related disorders are less likely to be physically active than those without. Conventional treatment may reduce inflammation and pain, but fail to stop progressive disease or deterioration. Analgesics may help to temporarily reduce pain and inflammation, but they have not been shown to repair damage. Other problems resulting from arthritis, osteoarthritis and/or rheumatoid arthritis and joint-related disorders are known to those of ordinary skill in the art.

Therefore, it is desirable to provide a compound, such as a dietary supplement, that addresses these problems and otherwise improves upon the healthy function of joints and connective tissue.

### SUMMARY OF THE INVENTION

In varying embodiments described herein, the invention relates to a compound that is capable of treating and mitigating the effects of arthritis and joint-related disorders. The unique combination of the composition is preferably administered orally in the form of a capsule. Methods for formulating the compound are also disclosed herein. The unique combination has synergistic advantages over previously known compositions.

As disclosed in more detail in the Detailed Description, the present invention provides both compositions and methods for treating arthritis and joint-related disorders. The composition is preferably comprised of a unique and novel formulation in pre-determined amounts, and further provides benefits previously unexpected. In addition to other health benefits described herein, the composition:
Reduces pain and inflammation;
Supports healthy joint structure and function;
Supports flexibility by improving lubrication of the joints;
Supports healthy glutathione production;
Soothes achy joints by reducing pro-inflammatory cytokines (pain-causing chemicals);
Cushions and lubricates sore achy joints; and
Supports healthy glutathione thereby improving detoxification.

In a preferred embodiment, the composition is substantially free of shellfish derivatives. Many formulas on the market today contain glucosamine and chondroitin, and these are frequently (not always) derived from shellfish. Due to severe shellfish allergies that are not always known to a person, a preferred embodiment of the formula would remain free of shellfish derivatives.

In a preferred embodiment, the composition is comprised of at least selenium, methylsulfonylmethane (MSM), boswellia, Type II collagen “collagen,” as well as hyaluronic acid and ginger. Other ingredients can be included as described herein.

More particularly, a preferred embodiment of the present invention comprises predetermined quantities of at least the following components: from about 10 mcg to 200 mcg of selenium; from about 200 mg to 1200 mg of MSM; from about 100 mg to 900 mg of boswellia; from about 1 mg to 5 mg collagen; from about 25 mg to 200 mg of hyaluronic acid; and from about 50 mg to 500 mg of ginger.

In an embodiment, the composition can be provided as a dietary supplement. In an embodiment, the composition can be administered in the form of a capsule. In another embodiment, the composition can be administered in the form of a tablet, as a suspension, sublingual spray, powder for a drink or dissolvable lozenge.

As used herein, the phrases “at least one”, “one or more”, and “and/or”, as used herein, are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about”.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof are meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

Advantages of the present invention will be apparent from the disclosure of the invention(s) contained herein. The above-described embodiments, objectives, and configurations are neither complete nor exhaustive. The Summary of the Invention is neither intended nor should it be construed as being representative of the full extent and scope of the present invention.

Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention and the Detailed Description, and no limitation as to the scope of the present invention is intended by either the inclusion or non-inclusion of elements in this Summary of the Invention. Additional aspects of the present invention will become more readily apparent from the Detailed Description.

### DETAILED DESCRIPTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

### Composition:

### Selenium

In a preferred embodiment, the composition includes a pre-determined amount of selenium. Consumption of Selenium promotes glutathione production, a major natural detoxificant and potent liver antioxidant. However, it can be depleted easily due in part to dietary consumption, for example, by drinking wine or taking medications, or eating poorly. Replenishment of glutathione is useful for optimal health.

Glutathione cannot be made without sufficient amounts of selenium. Selenium is a mineral antioxidant. More specifically, this antioxidant is essential for people with autoimmune arthritis (termed rheumatoid). Rheumatoid arthritis sufferers often have a selenium deficiency. It has been discovered through experimentation that selenium can help relieve arthritis symptoms by regulating free radical production and preventing damage to healthy tissues.

In addition, selenium can regulate immune function, which can account for selenium's beneficial effect in rheumatoid arthritis. Selenium intake has been found to eliminate joint pain, reduce joint stiffness, improve mobility, decrease joint inflammation, support healthy joints and lower the risk of developing arthritis, due in part to its ability to neutralize inflammatory free-radicals which lodge in joints. It has further been found that deficiency of selenium equates to a higher risk of joint pain and immobility.

In a preferred embodiment, the composition includes from about 10 mcg to 200 mcg of selenium. In a most preferred embodiment, the composition includes about 30 mcg of selenium.

### Collagen Type II

In a preferred embodiment, the composition further includes a pre-determined amount of type II collagen. Commonly referred to as “collagen,” it can be undenatured or denatured. Collagen is the principal structural protein in cartilage. Cartilage is a primary connective tissue in the body, providing flexibility and support to bones and joints, and confers tensile strength and toughness.

Collagen has been discovered through experimentation to be a desirable component of cartilage. The collagen can be derived from chicken sternum cartilage, collagen can be undenatured, which works with the immune system to support healthy joints and promote more mobility and flexibility. Collagen's composition has demonstrated its efficacy in human clinical studies. In a preferred embodiment, the composition includes a pre-determined amount of collagen. In an embodiment, the composition further includes a synergistic blend of anti-inflammatories, antioxidants and lubricating compounds.

In an October 2013 study in the Journal of The International Society of Sports Nutrition, it was found that a patented form of undenatured type II collagen could also reduce knee pain and improve mobility. See Lugo et al., Undenatured type II collagen (UC-II®) for joint support: A randomized, double-blind placebo-controlled study in healthy volunteers (incorporated by reference in its entirety). This research was based upon randomized, double-blind, placebo-controlled study in healthy volunteers, and concluded that daily supplementation of UC-II® was “well tolerated and led to improved knee joint extension” in the subjects. Adverse events related to the product were not observed during the study.

Accordingly, dietary supplements containing collagen have been proven to assist in reestablishing normal joint function. Collagen is also effective in supporting joint elasticity and reducing stiffness. A study in the International Journal of Clinical Pharmacology Research stated, “UC-II has demonstrated the ability to induce tolerance, effectively reducing joint pain and swelling in [rheumatoid arthritis] subjects.” Bagchi et al., Effects of Orally Administered Undenatured Type II chicken Collagen Against Arthritic Inflammatory Pathologies: A Mechanistic Exploration, 22(3-4):101-10 (2002) (incorporated by reference in its entirety). The authors of this study concluded, “UC-II [collagen] may serve as a novel therapeutic tool in joint inflammatory conditions and symptoms of [osteoarthritis] and [rheumatoid arthritis].” Id.

In a preferred embodiment, the composition includes from about 1 mg to 5 mg of type II collagen. In a most preferred embodiment, the composition includes about 2 mg of type II collagen.

### Boswellia

In a preferred embodiment, the composition further includes a pre-determined amount of boswellia extract. Boswellia extract is sometimes called Frankincense. It comes from the sap of the Boswellia serrata tree. Boswellia has been found to support joint health and reduce pain and inflammation by suppressing 5 lipoxygenase (5-lox). There are prescription medications on the market today that suppress this same pathway, and are termed “5-LOX inhibitors.” These medications, however, come with a variety of side effects.

Through experimentation it has been discovered that compounds including a pre-determined amount of Boswellia may help reduce pain or slow the production of inflammation chemicals. Thus, in a preferred embodiment, the composition includes from about 100 mg to 900 mg of boswellia. In a most preferred embodiment, the composition includes about 250 mg of boswellia.

### MSM (Methylsulfonylmethane)

In a preferred embodiment, the composition further includes a pre-determined amount of MSM. MSM is a sulfur containing compound that occurs naturally in certain plants and foods. It is included to provide sulfur to the joints and aid detoxification.

Sulfur has also been found to be beneficial to treating joint disease. Recent experimentation has shown that MSM (methylsulfonylmethane) supplementation may improve joint function, relieve symptoms associated with joint degeneration and improve mobility and quality of life among older individuals. Therefore, providing a composition that also includes appropriate levels of MSM can be beneficial.

Through experimentation, applicant has found that MSM shows positive effects in improving joint function, relieving symptoms associated with joint degeneration and improving the quality of life in aging populations. Several studies have shown significant benefit in arthritis, especially of the knee. In the publication, International Journal of Biomedical Science (available at http://www.ijbs.org/user/ContentFullText.aspx?VolumeNO=11&StartPage=54#ABS, last viewed Dec. 14, 2016 (incorporated in its entirety by reference)), a total of 100 male and female participants over 50 years old who had at least one of the related symptoms of joint degeneration (joint pain, joint stiffness, joint swelling, difficulty walking, difficulty getting up from bed and difficulty going down stairs) were recruited for a study. The participants were given MSM and monitored for symptoms of joint degeneration before and after the intervention, and the results were recorded. MSM was shown to improve joint function, relieve symptoms of joint degeneration and improve activities of daily living in the participants. Furthermore, MSM is required to provide a sulfur component which improves both joint structure and function. The clinical use of sulfur in the diet has been shown to be useful in optimizing health. Sulfur is needed to product the natural antioxidant glutathione, which neutralizes toxins in the body.

In a preferred embodiment, the composition includes from about 200 mg to 1200 mg of MSM. In a most preferred embodiment, the composition includes about 400 mg of MSM.

### Hyaluronic Acid

In a preferred embodiment, the composition further includes a pre-determined amount of the hyaluronic acid. Through experimentation, it has been determined that insufficiency of hyaluronic acid leads to greater pain. Hyaluronic acid can further be injected into knee joints as a temporary treatment for osteoarthritis.

Hyaluronic acid acts as a “lubricant” and helps arthritis sufferers feel better even if they must be supported on analgesic drugs, physical therapy or topical anesthetics. A recent 5-year study in Arthritis Research and Therapy (Sasaki et al., Serum hyaluronic acid concentration predicts the progression of joint space narrowing in normal knees and established knee osteoarthritis—a five-year prospective cohort study, 17:283 (2015) (incorporated by reference in its entirety)), concluded that there is a correlation in serum hyaluronic acid levels and the progression of knee joint deterioration. The less hyaluronic acid, the faster the decline, meaning the individual will likely be more apt to suffer pain, inflammation and inflexibility. With respect to joint function, hyaluronic acid help lubricate the joints, improving mobility and reducing pain.

In a preferred embodiment, the composition includes from about 25 mg to 200 mg of hyaluronic acid. In a most preferred embodiment, the composition includes about 50 mg hyaluronic acid.

### Ginger

Ginger is an herb known botanically as Zingiber officinale, and has strong medicinal benefits as an anti-inflammatory. Ginger is also thought to reduce nausea and vomiting. However the inclusion of this herb in this embodiment, and more particularly in the form of a ginger root extract, is for he reduction of pain-causing compounds that are released by the human body, specifically in connection with chronic pain syndrome. For example, the compound 5-LOX is known to be released within the body and can cause tremendous pain and inflammation. Ginger root extract has been shown to reduce this pain-causing compound. Other compounds such as tumor necrosis factor alpha (TNFα) and cyclooxygenase 2 (COX 2) are two other pain-causing compounds that rise in the human body during arthritic pain flares and chronic joint-related syndromes.

Ginger also has analgesic properties from its healing polysaccharides and flavonoids, which reduce inflammatory cytokines (by blocking NF-kB, a pathway in the body that secretes pain-causing compounds). Because of its ability to quell free radicals, ginger can also reduce the damage to joints by superoxides.

Reducing any of the above-mentioned pain-causing biomarkers (termed cytokines) in turn reduces pain and inflammation. As an added benefit, ginger root has been found to target and kill a harmful bacterium called H. pylori, (coma only referred to as the “ulcer bug” because it is frequently found in people with ulcers). Regretfully, this same pathogen, H. pylori has been implicated in the pathogenesis of joint inflammation. Eradicating H. pylori seems to be especially advantageous in infected rheumatoid arthritis patients. Ginger root reduces pro-inflammatory cytokines and helps to eradicate a destructive pathogen which may contribute to autoimmune arthritis (such as rheumatoid).

In a preferred embodiment, the composition includes from about 50 mg to 500 mg of ginger. In a most preferred embodiment, the composition includes about 180 mg of ginger.

In addition to the benefits described herein, several of the botanicals and nutrients described in varying embodiments of the present invention have been found to exhibit antifungal and antibacterial activity against a wide variety of organisms, which in turn provides an additional layer of protection to joints, cartilage and connective tissue, and may further reduce pain and inflammation.

### Additional Embodiments

In addition, the composition of a preferred embodiment can be substantially free of fish, shellfish, gluten, wheat, egg, peanuts, tree nuts, dairy, sugar, corn, soy, artificial colors and preservatives, or combinations thereof.

The composition can be in the form of a capsule, a tablet, a suspension, a sublingual spray, a powder for a drink or a dissolvable lozenge. The capsule can be vegetarian.

### Method of Treating a Patient

An aspect of the invention is a method to improve the function of joints and connective tissue. The method includes providing the patient with a composition, where the composition includes from about 10 to 200 mcg of selenium, from about 1 to 5 mg of collagen, from about 100 to 900 mg of boswellia, from about 200 to 1200 mg of MSM, from about 25 to 200 mg of hyaluronic acid, and from about 50 to 500 mg of ginger. Other components such as a palatability agent can also be added to the composition. Between about 386 mg and about 3005 mg of the composition can be provided to the patient. The composition can be provided in the form of a capsule, a tablet, a suspension, a sublingual spray, a powder, or a lozenge. Between one to four capsules can be provided to a patient per day. The capsule can be size 0, which corresponds to an overall closed length of between about 20.7-21.7 mm, and an average weight of between 98-106.4 mg +/−6%. The number of capsules can be sized to provide the patient with between about 386 mg and about 3005 mg per day. As the number of capsules vary, the size of the capsule can vary as well.

An aspect of the invention is a treatment for a patient to improve function of the joints and connective tissue by administering the composition of the present invention. Another aspect of the invention is a method to regenerate connective tissue in a patient by administering the composition of the present invention.

### Method of Making the Composition

An aspect of the invention is a method to prepare a composition. The method comprises providing proportional amounts of each material such that the resulting composition results containing from about 10 to 200 mcg of selenium, from about 1 to 5 mg of collagen, from about 100 to 900 mg of boswellia, from about 200 to 1200 mg of MSM, from about 25 to 200 mg of hyaluronic acid, and from about 50 to 500 mg of ginger. Other components such as a palatability agent can also be added to the composition. The components are mixed, then can be provided to a delivery device (for example capsule, tablet, suspension, sublingual spray, powder for a drink or dissolvable lozenge).

Ranges have been discussed and used within the forgoing description. One skilled in the art would understand that any sub-range within the stated range would be suitable, as would any number within the broad range, without deviating from the invention.

While various embodiment of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

What is claimed is:

1. A composition for treating arthritis and joint-related syndromes comprising selenium, MSM, boswellia, collagen (type II), hyaluronic acid, and ginger.

2. The composition of claim 1, further comprising from about 10 to 200 mcg of the selenium, from about 200 mg to 1200 mg of the MSM, from about 100 to 900 mg of the boswellia, from about 1 to 5 mg of the collagen (type II), from about 25 to 200 mg of the hyaluronic acid, and from about 50 to 500 mg of the ginger.

3. The composition of claim 1, wherein the composition is provided as a dietary supplement.

4. The composition of claim 1, wherein the composition is administered orally in the form of a capsule.

5. The composition of claim 1, wherein the composition is administered orally in the form of a tablet.

6. The composition of claim 1, wherein the composition comprises
about 30 mcg of selenium;
about 2 mg of collagen;
about 250 mg of boswellia;
about 400 mg of MSM;
about 50 mg hyaluronic acid; and
about 180 mg of ginger.

7. The composition of claim 1, wherein the composition does not include at least one of a fish, a shellfish, a gluten, a wheat, an egg, a peanut, a tree nut, dairy, a sugar, a corn, soy, an artificial color, or a preservative.

8. A method to make a composition, comprising:
mixing selenium, MSM, boswellia, type II collagen, hyaluronic acid, and ginger to produce a composition; and
forming the composition into a delivery device, wherein the delivery device is in the form of a capsule, a tablet, a suspension, a sublingual spray, a powder or a lozenge.

9. The method of claim 8, further comprising from about 10 to 200 mcg of the selenium, from about 200 mg to 1200 mg of the MSM, from about 100 to 900 mg of the boswellia, from about 1 to 5 mg of the type II collagen, from about 25 to 200 mg of the hyaluronic acid, and from about 50 to 500 mg of the ginger.

10. The method of claim 8, comprises:
about 30 mcg of selenium;
about 2 mg of type II collagen;
about 250 mg of boswellia;
about 400 mg of MSM;
about 50 mg hyaluronic acid; and
about 180 mg of ginger.

11. The method of claim 8, wherein the method does not include providing at least one of a fish, a shellfish, a gluten, a wheat, an egg, a peanut, a tree nut, dairy, a sugar, a corn, soy, an artificial color, or a preservative to the mixture

12. A method to improve joint function in a patient, comprising:
providing between 386 mg and 3005 mg of a composition to a patient, wherein the composition comprises selenium, collagen, boswellia, MSM, hyaluronic acid, and ginger.

13. The method of claim 12, further comprising from about 10 to 200 mcg of the selenium, from about 200 mg to 1200 mg of the MSM, from about 100 to 900 mg of the boswellia, from about 1 to 5 mg of the type II collagen, from about 25 to 200 mg of the hyaluronic acid, and from about 50 to 500 mg of the ginger.

14. The method of claim 12, comprising:
about 30 mcg of selenium;
about 2 mg of type II collagen;
about 250 mg of boswellia;
about 400 mg of MSM;
about 50 mg hyaluronic acid; and
about 180 mg of ginger.

15. The method of claim 12, wherein the composition is provided to the patient in the form of a capsule, a tablet, a suspension, a sublingual spray, a powder, or a lozenge.

16. The method of claim 15, wherein the composition is provided in the form of the capsule or the tablet, and wherein between one and four of the capsule or the tablet is provided to the patient.

17. The method of claim 12, wherein the joint function is improved by regenerating connective tissue.

18. The method of claim 12, wherein a diminished joint function in the patient is caused by arthritis.
